(19) United States
(12) Patent Application Publication
(10) Pub. No.: US 2004/0204661 A1
(43) Pub. Date: Oct. 14, 2004

(54) Genomics home test kit

(76) Inventor: Gary R. Epler, Wellesley, MA (US)

Correspondence Address: MESMER & DELEAULT, PLLC, 41 BROOK STREET, MANCHESTER, NH, 03104, US

(21) Appl. No.: 10/411,459

(22) Filed: Apr. 10, 2003

(51) Int. Cl. A61B 10/00

(52) U.S. Cl. 600/572

(57) ABSTRACT

A genomics home test kit has a sample container having two absorbent sample holding pads impregnated with chemicals to lyse cell membranes and immobilize nucleic acids, a pair of buccal cell sampling swabs and instructions for collecting, applying and submitting a patient's buccal cell sample for genomic analysis to customize a medical therapy. The first holding pad has a first indicia and the second holding pad has a second indicia. The first sampling swab has indicia linking the first sampling swab to the first holding pad. The second sampling swab has indicia linking the second sampling swab to the second holding pad.

### BACKGROUND OF THE INVENTION

1. Field of the Invention

The present invention relates generally to medical diagnostic test kits.

2. Description of the Prior Art

Numerous medical test are commercially available for use in the home. Two illustrative examples include medical test kits that are used to discover an individual's blood sugar, i.e. glucose, level at a particular time, or an individual's hormone level at a particular time.

The glucose test kit allows a diabetic to test his/her blood sugar and adjust his/her daily insulin dosages accordingly without consulting a doctor or other medical personnel. To initially learn to take blood and use a personal glucose test kit, a diabetic may get trained by a nurse or other medical personnel. Not all personal test kits require the taking of blood or other internal body fluid. For example, personal pregnancy test kits are commercially available that allows a woman, in the privacy of her own home, to test her urine for the presence of a pregnancy hormone, human chorionic gonadotropin.

DNA test kits that are used to determine familial relationships are also commercially available. DNA test kits use DNA sequencing technology to determine the familial relationship of an individual. DNA sequencing is the determination of the order of nucleotides (the base sequence) that exists in a DNA molecule of an individual.

As one example of DNA lineage testing, GeneTree™ offers DNA Personal Paternity Tests. DNA Personal Paternity Tests can be used in the home to statistically determine the likelihood of a particular man being a child's father. The GeneTree™ DNA Personal Paternity Tests utilize cheek cells collected with colored swabs where one color indicates an alleged father and a second color indicates the subject child. The colored swabs are placed in coordinating colored envelopes that match the color of the user's swab. The envelopes are then sent to a laboratory where genetic testing is performed according to standard procedures.

Unfortunately, most medical test kits on the market today are used for determining events that have already occurred. These test kits determine the existence of a condition such as low blood sugar, pregnancy, etc., or to determine the statistical probability of a genetic link between two or more individuals.

There exists today a problem with the system used for the development of new drugs and drug treatments. Currently, pharmaceutical companies are limited to developing drugs using a one-size-fits-all system. This system allows for the development of drugs to which the average patient will respond. Unfortunately, some patients have a severe negative reaction to a prescribed drug. In the industry, this is called an adverse drug reaction.

A 1998 study of hospitalized patients published in the Journal of the American Medical Association reported that in 1994, adverse drug reactions accounted for more than 2.2 million serious cases and over 100,000 deaths. An adverse drug reaction is one of the leading causes of hospitalization and death in the United States. Currently, there is no simple way to determine if people will respond well, badly or not at all to a medication.

With the advent of the Internet and the proliferation of medical-related information, patients are becoming more aware of the seriousness of adverse drug reactions. They may even know someone who has suffered such an event. Harvard Business School Professor Regina Herzlinger, writing in the July 2002 issue of the Harvard Business Review, reports that patients are demanding better, more tailored treatments. The article further notes that a report in the Journal of the American Medical Association showed that screening drugs against a person's genetic makeup could reduce many dangerous reactions. The AMA report revealed that more than half of the 27 drugs frequently cited for causing adverse reactions were linked to genetic variations in a patient's ability to metabolize the drugs.

Therefore what is needed is a minimally-invasive test kit that can be used without the assistance of medical personnel. What is further needed is a test kit that can be used in the user's home while preventing contamination of the sample. What is also needed is a test kit that is used to predict a patient's potential of an adverse medical reaction to a particular drug treatment. What is even further needed is a test kit that is used to predict the efficacy of a particular medication within the user's body.

### SUMMARY OF THE INVENTION

It is an object of the present invention to provide a diagnostic genomics home test kit. It is also an object of the present invention to provide a simple genomics home test kit that can be utilized by any individual. It is further an object of the present invention to provide a minimally-invasive genomics home test kit that can be used without the assistance of medical personnel. It is an even further object of the present invention to provide a genomics home test kit that can be used in the user's home while preventing contamination of the sample. It is still a further object of the present invention to provide a genomics home test kit that will predict the likelihood of a patient's adverse medical reaction to a particular drug treatment. It is another object of the present invention to provide a genomics home test kit that will predict the efficacy of medications within the user's body. It is yet another object of the present invention to provide a genomics home test kit for determining the likelihood of potential liver dysfunction in individuals as well as cardiac toxicity, pulmonary toxicity and other organ toxicity.

The present invention achieves these and other objectives by providing a personal genomics home test kit with a sample container comprising a hinged folder having a first and a second sample holding pad, a first and a second cell collection device corresponding to the first and second sample holding pads, and an instruction sheet. The genomics test kit may also include a desiccant pillow and an outer pouch sized to contain both the utilized sample container and desiccant pillow.

The hinged folder of the sample container has a first surface with locations for the first and second sample holding pads, and a second surface that overlays the first surface. The sample holding pads of the sample container first surface are protected by the second surface. The sample container first surface has indicia markings below the first and second sample holding pads. The indicia markings indicate the proper location for placing a first and a second cell sample upon the appropriate sample holding pad. The first and second surfaces of the sample container are approximately of equal size.

The second surface may have an extended portion, i.e. a securing tab, for securing the second surface to the first surface. The securing tab may either fold over a lower portion end of the first surface or may be inserted into a slit of the first surface below the first and second sample holding pads. The securing tab may also have a pressure-sensitive adhesive to secure the tab against the back side of the first surface of the sample container near the lower portion end of the first sample container surface below the first and second sample holding pads. Alternatively, the second surface may have a coating of a pressure-sensitive adhesive along a portion adjacent an edge of the second surface for adhering to the first surface.

The sample holding pads on the first surface of the sample container are sized and shaped to allow enough quantity of cell sample to achieve accurate results for the desired pharmacogenomic use. In this instance, it is to determine the presence of risk markers in a person's DNA that predicts a high probability of possible liver dysfunction or other organ dysfunction leading to an adverse drug reaction. First and second sample holding pads may be any shape and may each be a different shape. The sample holding pads are absorbent and impregnated with chemicals to lyse cell membranes on contact, to immobilize and stabilize nucleic acids (DNA and RNA), and to inactivate bacteria and viruses. The sample holding pads allow biological cell samples to be collected, transported and stored at room temperature.

The first and second cell collection devices specifically correspond to the first and second sample holding pads. The first and second cell collection devices are generally flat, elongated rectangular swabs composed of an inert, i.e. non-reactive, material that will not introduce contaminants onto the sample holding pad. The cell collection devices have indicia markings on both sides that indicate the correct location of finger placement to pick up and utilize the device, and the location of cell collection. The indicia for locating finger placement is important to prevent inadvertent sample contamination to the first and second cell collection locations on the cell collection device. Examples of contaminants that can damage the cell collection location include finger oils and dirt, hand soap and hand lotion residues, fingernail polishes and other cosmetic product residues commonly found on the fingers or on the hand of a home user. The cell collection locations of the cell collection devices may also contain outer protective wrappings to protect the cell collection locations from external contamination until the user understands the instructions and is ready to proceed with cell collection.

The instruction sheet of the genomics home test kit may be either a separate sheet within the kit or may be an integral part of one of the surfaces of the sample container. For instance, the instructions may be printed on the outside surface of the foldable sample container. The instructions provide a sequence of detailed informative steps teaching the proper use of the genomics home test kit to achieve accurate results. For example, detailed information about the proper method of picking up the first cell collection device, obtaining a sample from the inside surface of a user's first cheek, placing the cell sample onto the indicia coordinated first holding pad of the sample container, and repeating the procedure for the same first cheek using the reverse side of the first collection device. Next, the previously described procedure is then followed for obtaining a sample for the inside surface of the user's second cheek using the second cell collection device and transferring the sample to the second holding pad of the sample container. Should a user be left-handed instead of right-handed, additional instruction about changes to the method of picking up and using a first and second cell collection device for use by a left-handed user are given. If the cell collection locations on the cell collection devices contain outer protective wrappings, the instruction sheet would also contain additional instructions about the proper time of removal and method of removal of the outer protective wrapping before cell collection. The additional instructions about the outer protective wrapping would be inserted at the appropriate location within the sequence of instructions.

An outer pouch of the genomics test kit is sized to contain the sample container, which has coded indicia markings that indicate the identity of the user of the kit. The coded indicia markings may be used for several purposes such as, for example, maintaining user privacy, maintaining correct sample identity during preliminary sample preparation and analysis by the testing laboratory, and returning the specific genomic test results to the correct user.

A desiccant pillow of the genomics test kit may be included to maintain a relatively dry atmosphere within the outer pouch. The desiccant pillow includes a material for absorbing excess moisture from the atmosphere in the pouch. This prevents moisture from adversely affecting the cell samples that are disposed onto the sample holding pads located inside the folded sample container.

The genomics home test kit incorporates indicia to alert a user that the kit is used to identify risk markers for predicting the probability of an adverse drug reaction. Particularly, it is the risk markers associated with the cytochrome P450 enzyme family. The cytochrome P450 enzymes are used in the metabolic processing of medication. Cytochrome P450 enzymes include the CYP3A gene family and CYP2D6. Identification of a user's risk markers is important because adverse drug reactions can result in liver dysfunction in some individuals. The risk markers are identified by examination of an individual's structural genes. In addition to the examination of the structural gene, genotypic testing of the regulatory/promoter regions, certain transcription factors and the gene sequences important for correct splicing will be performed. By this examination, the risk of dangerously large changes in liver function will be determined. By knowing what risk markers are present, the genomic analysis can be correlated to an effective drug therapy that minimizes the potential for an adverse drug reaction.

In order to maintain user privacy, a peelable label containing a tracking indicia is removably affixed to the back side of the sample container. The sample container also includes matching tracking indicia to that on the peelable label. Each genomics home test kit has a unique tracking indicia.

### BRIEF DESCRIPTION OF THE DRAWINGS

FIG. 1 is a perspective view of one embodiment of the present invention.

FIG. 2 is a top plan view of another embodiment of the present invention.

FIG. 3 is a top plan view of the present invention showing different shaped sample holding pads.

FIG. 4 is a back plan view of the present invention showing the tracking indicia.

FIGS. 5A and 5B are enlarged front plan views of the present invention showing the indicia on the buccal cell swabs.

### DETAILED DESCRIPTION OF THE PREFERRED EMBODIMENT

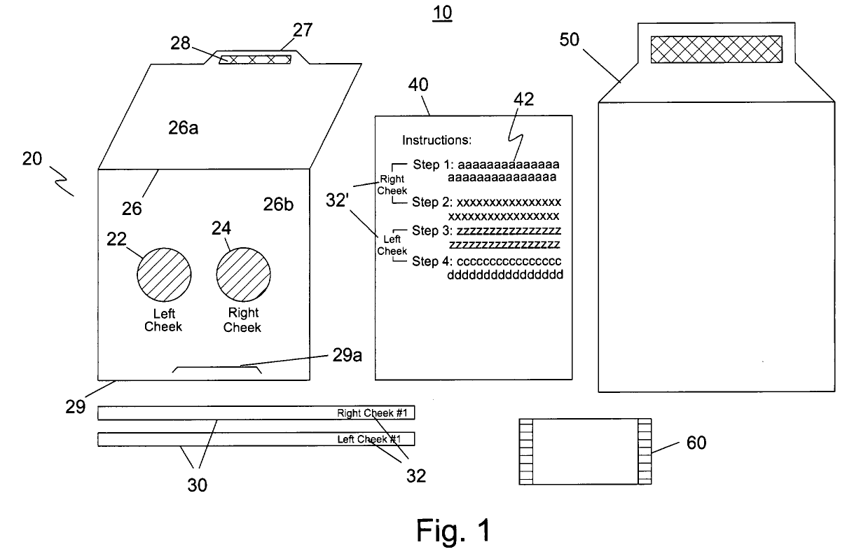

The preferred embodiment(s) of the present invention are illustrated in FIGS. 1-5. FIG. 1 illustrates front planar views of the genomics home test kit 10. The genomics home test kit 10 is specific for determining the presence of adverse drug reaction risk markers in a user's DNA. Genomics home test kit 10 includes a sample container 20, a pair of buccal cell swabs 30, instruction sheet 40, an outer pouch 50, and a desiccant 60. Sample container 20 includes a first sample holding pad 22 and a second sample holding pad 24. Sample container 20 is generally a hinged folder having a creased fold 26. Creased fold 26 divides sample container 20 into an upper portion 26a and a lower portion 26b of approximately equal size. Upper portion 26a has a securing tab 27 that may either fold over lower portion end 29 of lower portion 26b or inserted into a lower portion slit 29a. Securing tab 27 may also have a pressure-sensitive adhesive 28 to secure tab 27 against the back side (not shown) of lower portion 26b near lower portion end 29.

Sample holding pads 22 and 24 are generally impregnated with chemicals to lyse cell membranes and immobilize nucleic acids. Buccal cell swabs 30 are used for obtaining a buccal cell sample. Typically, buccal cell swabs 30 are made of wood to scrape a sample of epithelial cells from the inside cheek of a user. It is particularly important to include indicia 32 on each of cell swabs 30 to prevent sample contamination and to increase the probability of proper user compliance. Swabs 30 include indicia 32 on each side of the swab. Indicia 32 link the test sample to be collected with the associated sample holding pad 22 or 24.

Instruction sheet 40 includes instructions 42 that provide a sequence of detailed informative steps instructing in the proper use of the test kit 10 to achieve accurate adverse drug reaction risk marker test results. The instructions include the use of linking indicia 32′ to guide the user in use of swabs 30 and proper sample collection and transfer techniques as well as disclosing the type of test kit, i.e. to identify in a person's DNA the presence of risk markers that increase the probability of a particular drug causing liver dysfunction and leading to an adverse drug reaction. Instructions 42 provide detailed information about picking up the first swab or cell collection device 30, obtaining a sample from the inside surface of a user's first cheek and placing the obtained sample onto the proper holding pad 22, 24 as identified by the indicia 32 and 32′.

Outer pouch 50 is a sealable container sized to receive sample container 20 for transfer to another location. Desiccant 60 is a standard moisture-absorbent pillow sized for placement within outer pouch 50 along with sample container 20. Desiccant 60, when placed within outer pouch 50 with sample container 20, maintains a relatively dry atmosphere within outer pouch 50. The desiccant material is typically a substance having a high affinity to water molecules that binds and holds the water molecules found in the atmosphere within outer pouch 50.

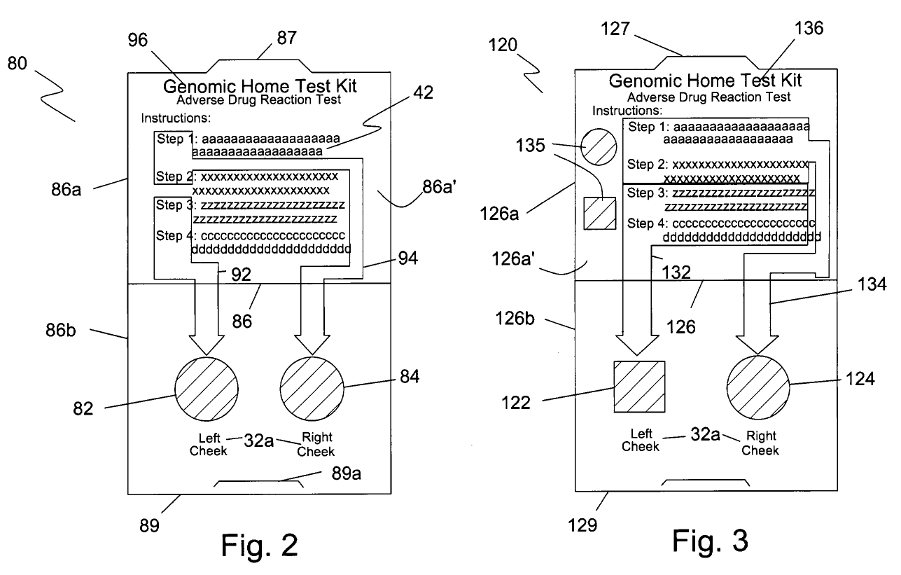

Turning now to FIG. 2, there is shown another embodiment of the sample container of the present invention. Sample container 80 includes a first sample holding pad 82 and a second sample holding pad 84. Sample container 80 is generally a hinged folder having a creased fold 86. Creased fold 86 divides sample container 80 into an upper portion 86a and a lower portion 86b of approximately equal size. Upper portion 86a has a securing tab 87 that may either fold over lower portion end 89 of lower portion 86b or inserted into a lower portion slit 89a.

In this embodiment, instructions 42 are imprinted on a second surface 86a′ of upper portion 86a. Indicating indicia 92 and 94 links the sampling instructions 42 for first and second cheek samples with the properly identified first and second holding pads 82, 84. Sample container 80 also has indicia 96 clearly identifying the adverse drug reaction risk marker kit.

FIG. 3 shows yet another embodiment of the sample container of the present invention. Sample container 120 includes a first sample holding pad 122 and a second sample holding pad 124. Sample container 120 is generally a hinged folder having a creased fold 126. Creased fold 126 divides sample container 120 into an upper portion 126a and a lower portion 126b of approximately equal size. Upper portion 126a has a securing tab 127 that may either fold over lower portion end 129 of lower portion 126b or insert into a lower portion slit 129a.

Like the embodiment in FIG. 2, instructions 42 are imprinted on a second surface 126a′ of upper portion 126a. Indicating indicia 132 and 134 may also connect the sampling instructions 42 for right and left cheek samples with the properly identified first and second holding pads 122, 124. Additional linking indicia 135 may be used to further link relevant portions of instructions 42 to sample holding pads 122 and 124. Sample container 120 also has indicia 136 clearly identifying the adverse drug reaction risk marker kit. Sample container 120 further includes a first holding pad 122 that has a different shape than second holding pad 124. In this example, the first holding pad 122, labeled Left Cheek, has a square shape. Second holding pad 124, labeled Right Cheek, has a circular shape. This shape differentiating indicia 135 is also included on swabs 30, which further helps the user in reducing contamination by visually connecting the additional indicating indicia to the swabs 30 for use with the proper holding pad 122 or 124.

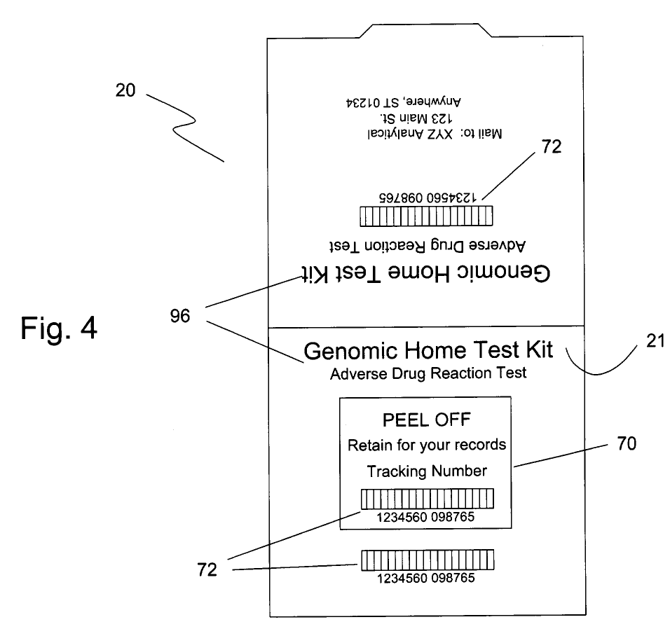

FIG. 4 is a back view of sample container 20. On a back surface 21, there is imprinted indicia 96 to indicate the type of test, indicia 72 for tracking the sample, a mailing address of the place for analysis, and a removable, peel-off sticker 70. Removable sticker 70 includes tracking indicia 72 for the user to obtain the results of the test while maintaining user privacy.

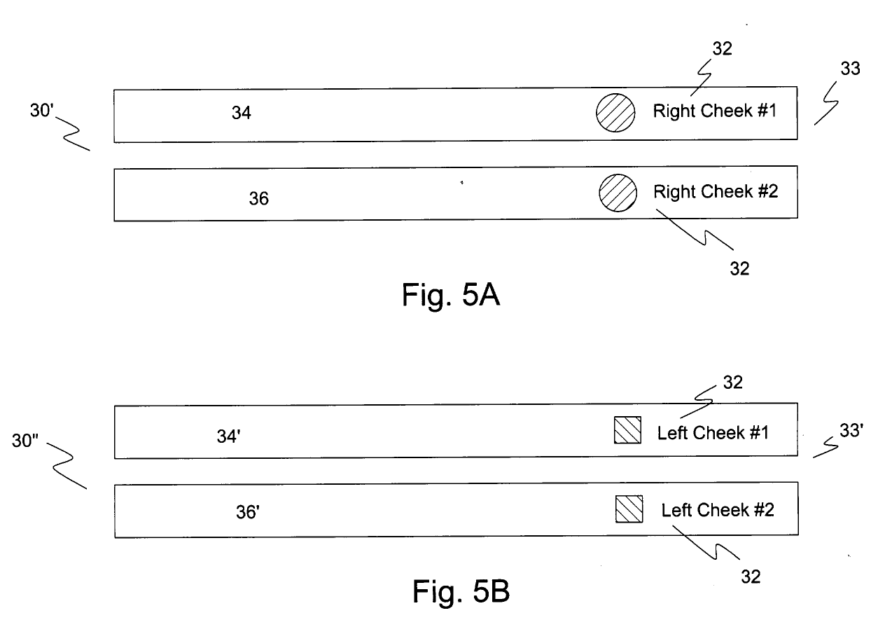

FIGS. 5A and 5B illustrate the indicating indicia on swabs 30. Turning now to FIG. 5A, right cheek swab 30′ has a first swab side 34 and a second swab side 36. Swab sides 34, 36 have imprinted thereon adjacent a swab end 33, indicia 32 indicating the sample holding pad associated with right cheek swab 30′. In this particular example both the shape and the word symbols indicate the proper sample holding pad for receiving the right cheek sample. In FIG. 5B, left cheek swab 30″ also has a first swab side 34′ and a second swab side 36′. Adjacent a swab end 33′, indicia 32 are imprinted to indicate the sample holding pad associated with left cheek swab 30″.

To use the genomics home testing kit 10, the user opens kit 10 and removes swabs 30 that have indicating indicia 32 on each side. In this case, indicating indicia 32 are “Right Cheek #1,” “Right Cheek #2,” “Left Cheek #1,” and “Left Cheek #2.” The user takes swab 30 marked “Right Cheek #1,” places the user's thumb over the words that say “Right Cheek #1,” and puts the swab into the user's mouth placing the flat surface against the inside of the right cheek. The user then gently rubs the tip of the swab up and down five times with a motion of about one-half of an inch. This motion results in a few cells lining the inside of the right cheek to stick to swab 30.

The user removes swab 30 from the mouth and swirls the tip of swab 30 containing the cells about ten times onto the second holding pad 24 marked “Right Cheek.” Using the same swab 30, the user is instructed to repeat the process by now placing the user's thumb over the words that say “Right Cheek #2.” After rubbing the tip of swab 30 against the inside of the right cheek, the user swirls the tip of swab 30 containing more cells about ten times onto the first holding pad 24. This process is repeated for the left cheek using swab 30 marked “Left Cheek #1” and “Left Cheek #2.”

Upon completion of sample collection, the sample container 20 is sealed by folding upper portion 26a over lower portion 26b and securing upper portion 26a to lower portion 26b using securing tab 27. The user then removes the peel off tracking number and retains it for reference purposes, and mails the sample container 20 to the indicated address for analysis.

Although the preferred embodiments of the present invention have been described herein, the above description is merely illustrative. Further modification of the invention herein disclosed will occur to those skilled in the respective arts and all such modifications are deemed to be within the scope of the invention as defined by the appended claims.

What is claimed is:

1. A genomics home test kit for customizing a medical therapy to a patient, said genomics home test kit comprising:
a sample container comprising a hinged folder having two absorbent sample holding pads impregnated with chemicals to lyse cell membranes and immobilize nucleic acids wherein a first holding pad has a first indicia and a second holding pad has a second indicia, said pads configured for receiving a patient's buccal cell sample;
a pair of buccal cell sampling swabs wherein a first swab of said sampling swabs has indicia linking said first swab to said first indicia and a second swab of said sampling swabs has indicia linking said second swab to said second indicia; and
instructions for collecting, applying and submitting said patient's buccal cell sample for customizing a medical therapy using genomic information derived from said buccal sample of said patient.

2. The system of claim 1 wherein said linking indicia is a name, a symbol, a graphic feature or a structural feature and combinations thereof.

3. The system of claim 1 further comprising an outer pouch for receiving said sample container.

4. The system of claim 3 further comprising a desiccant to be added to said outer pouch.

5. An over-the-counter medical therapy customizing system comprising:
a sampling card having at least a first and a second absorbent sample holding pads wherein said first and second holding pads are impregnated with chemicals to lyse cell membranes and immobilize nucleic acids, said pads configured for receiving a patient's buccal cell sample;
at least a first pad indicia associated with said first holding pad;
at least a second pad indicia associated with said second holding pad;
at least a first buccal cell swab having a first swab indicia linked to said first pad indicia;
at least a second buccal cell swab having a second swab indicia linked to said second pad indicia; and
instructions for collecting, applying and submitting said patient's buccal cell sample for customizing a medical therapy using genomic information derived from said buccal cell sample of said patient.

6. The system of claim 5 wherein said first pad indicia, said second pad indicia, said first swab indicia, and said second swab indicia are a name, a symbol, a graphic feature or a structural feature and combinations thereof.

7. The system of claim 5 wherein said instructions are printed on said sampling card.

8. The system of claim 7 wherein said instructions further include linking indicia linking portions of said instructions to said first and second sample holding pads.

9. The system of claim 7 wherein said sampling card has a peelable tracking indicia and identical permanent tracking indicia.

10. A method for customizing the selection of medical therapies for a patient, said method comprising:
obtaining a buccal cell sample from said patient;
disposing said buccal cell sample onto at least one absorbent sample holding pad of a sample container, said holding pad treated with one or more chemicals to lyse cell membranes and immobilize nucleic acids;
performing a genomic analysis on said buccal cell sample to determine the presence of risk markers in DNA of said cell sample relevant to predicting an adverse drug reaction to a predefined drug; and
determining an effective drug therapy based on said genomic analysis.

11. The method of claim 10 further comprising providing instructions for collecting, applying and submitting said buccal cell sample of said patient.

12. The method of claim 11 further comprising providing linking indicia on portions of said instructions that associate said portions to said at least one sample pad.

13. The method of claim 10 further comprising providing a peelable tracking indicia that is identical to tracking indicia printed on said sample container.

14. A method to improve the effectiveness of a drug therapy for a patient, said method comprising:
providing a home test kit for obtaining a buccal cell sample from said patient,
said kit comprising:
a sampling card having at least a first and a second absorbent sample holding pads wherein said first and second holding pads are impregnated with chemicals to lyse cell membranes and immobilize nucleic acids, said pads configured for receiving said patient's buccal cell sample;
at least a first pad indicia associated with said first holding pad and at least a second pad indicia associated with said second holding pad;
at least a first buccal cell swab having a first swab indicia linked to said first pad indicia and at least a second buccal cell swab having a second swab indicia linked to said second pad indicia; and
instructions for collecting, applying and submitting said patient's buccal cell sample;
performing a genomic analysis on said patient's buccal cell sample to determine the presence of risk markers on the DNA of said cell sample linked to adverse drug reactions; and
recommending a medical therapy for said patient based on said genomic analysis.

15. The method of claim 14 further comprising providing linking indicia on portions of said instructions that associate said portions to said at least a first and second sample holding pads.

16. The method of claim 14 further comprising providing a peelable tracking indicia that is identical to tracking indicia printed on said sampling card.

17. A system for improving the effectiveness of a drug therapy for a patient, said system comprising:
a buccal cell sample collecting means;
a buccal cell sample receiving means for receiving a buccal cell sample from a patient obtained using said buccal cell sample collecting means;
instruction means for disclosing to said patient the procedure for collecting, applying and submitting said patient's buccal cell sample for genomic analysis; and
correlation means for correlating said genomic analysis of said patient's buccal cell sample to an effective drug therapy for said patient for a particular treatment regimen.

18. The system of claim 17 wherein said buccal cell sample receiving means has first and second absorbent sample holding pads wherein said first and second holding pads are impregnated with chemicals to lyse cell membranes and immobilize nucleic acids.

19. The system of claim 17 further comprising at least a first pad indicia associated with said first absorbent sample holding pad and at least a second pad indicia associated with said second absorbent sample holding pad.

20. The system of claim 19 wherein said buccal cell sample collecting means is at least a first buccal cell swab having a first swab indicia and at least a second buccal cell swab having a second swab indicia means is a name, a symbol, a graphic feature or a structural feature and combinations thereof.
